(19) United States
(12) Patent Application Publication
(10) Pub. No.: US 2021/0321954 A1
(43) Pub. Date: Oct. 21, 2021

(54) SYSTEM AND METHOD FOR NETWORK ANALYSIS OF A PATIENT'S NEURO CARDIO-RESPIRATORY-SYSTEM

(71) Applicant: LOUISIANA TECH RESEARCH CORPORATION, Ruston, LA (US)

(72) Inventors: Leonidis Iasemidis, Ruston, LA (US); Timothy N. Hutson, Ruston, LA (US)

(21) Appl. No.: 17/271,961

(22) PCT Filed: Sep. 3, 2019
(86) PCT No.: PCT/US2019/049313

§ 371 Date: Feb. 26, 2021

Related U.S. Application Data
(60) Provisional application No. 62/725,483, filed on Aug. 31, 2018.

(51) Int. Cl. A61B 5/00; G16H 50/20; G16H 50/30; A61B 5/0245; A61B 5/021

(52) U.S. Cl. CPC A61B 5/7275; A61B 5/4035; A61B 5/4064; A61B 5/7282; G16H 50/20; G16H 50/30; A61B 2560/02; A61B 5/021; A61B 5/0035; A61B 5/4094; A61B 5/4082; A61B 5/4818; A61B 5/0245

(57) ABSTRACT

Network analysis of a patient's neuro-cardio-respiratory system can be performed to detect an impending crisis, diagnose an abnormality, and/or provide informative feedback about a treatment regime. A system can receive data recorded by one or more recording devices from a patient. The data is time varying and related to two or more organs of the patient's neuro-cardio-respiratory system that are monitored. The system can estimate directional interactions between the two or more organs within the patient's body over time via a mathematical analysis of the data to identify one or more pathologies in a network of the neuro-cardio-respiratory system. When the one or more pathologies are identified, the system can provide the advance warning of the impending crisis, the diagnosis of the abnormality, or the informative feedback for the treatment regime.

### RELATED APPLICATIONS

This application claims the benefit of U.S. Provisional Application No. 62/725,483, filed Aug. 31, 2018, entitled “DIRECTIONAL INFORMATION FLOW FOR DIAGNOSIS OF SUSCEPTABILITY TO NEUROCARDIOVASCULAR CRISES AND DISEASES,” the entirety of which is hereby incorporated by reference for all purposes.

### GOVERNMENT SUPPORT

This invention was made with U.S. government support under OIA 1632891 awarded by the National Science Foundation. The government has certain rights in this invention.

### TECHNICAL FIELD

The present disclosure relates to network analysis of a patient's neuro-cardio-respiratory system and, more specifically, to systems and methods for detecting an impending crisis, diagnosing an abnormality, and/or providing informative feedback about a treatment regime via network analysis of the patient's neuro-cardio-respiratory system.

### BACKGROUND

A patient's neuro-cardio-respiratory system includes tightly-coupled interdependent organs (e.g., two or more of a patient's brain, heart, lungs, muscle, vasculature, etc.) that perform functions vital to a patient's life. Loss of function in any of the interdependent organs can lead to severe consequences (e.g., an epileptic seizure can interrupt communication from the brain to the heart and/or lungs, which may lead to sudden unexplained death in epilepsy (SUDEP)). While anatomical and physiological connections between the organs are informative, there are not many ways to measure such structural connections in real time or to infer the influence of one organ on another. A more effective measure of coupling and influence is to analyze simultaneous signals from each of the organs in the system and infer their functional connectivity.

### SUMMARY

The present disclosure relates to network analysis of a patient's neuro-cardio-respiratory system, which analyzes simultaneous signals from each of the organs in the system. The organs can be modeled as parts of a unified and directed network of the system. Based on this analysis, the systems and methods of the present disclosure can detect an impending crisis, diagnose an abnormality, and/or provide informative feedback about a treatment regime.

In one aspect, the present disclosure includes a system that performs a network analysis of a patient's neuro-cardio-respiratory system. The system includes a non-transitory memory storing computer-executable instructions; and a processor that executes the computer-executable instructions. Upon execution of the computer-executable instructions, the system can receive time varying data related to two or more organs of the neuro-cardio-respiratory system within the patient's body recorded by one or more recording devices from the patient; estimate directional interactions between the two or more organs within the patient's body over time via a mathematical analysis of the acquired data to identify one or more pathologies in a network within the neuro-cardio-respiratory system; and provide an advance warning of an impending crises, a diagnosis of the abnormality, or informative feedback for a treatment regime when the one or more pathologies in the network of neuro-cardio-respiratory system are identified.

In another aspect, the present disclosure includes a method for performing a network analysis of a patient's neuro-cardio-respiratory system. At least a portion of the method can be performed by a system comprising a processor. Data recorded by one or more recording devices from the patient can be received. The data is time varying and related to two or more organs of the neuro-cardio-respiratory system within the patient's body that are monitored. Directional interactions between the two or more organs within the patient's body over time can be estimated via a mathematical analysis of the data to identify one or more pathologies in a network within the neuro-cardio-respiratory system. In response to identification of the one or more pathologies in the network of the neuro-cardio-respiratory system an advance warning of the impending crisis, a diagnosis of the abnormality, or informative feedback for a treatment regime can be provided.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a schematic block diagram showing an example of a system that performs a network analysis of a patient's neuro-cardio-respiratory system;

FIG. 2 is a schematic block diagram showing an example of the estimation unit shown in FIG. 1;

FIG. 3 is a process flow diagram showing an example of a method for performing a network analysis of a patient's neuro-cardio-respiratory system;

FIG. 4 is a process flow diagram showing an example method for estimating directional interactions between two or more organs;

FIG. 5 is an example of interactions occurring in the neuro-cardio-respiratory system;

FIG. 6 is a process flow diagram of another example method for estimating directional interactions between two or more organs by GPDC;

FIG. 7 shows the mean (points) and standard deviation (bars) of GPDC values per frequency band for ECG->EEG, EEG->ECG, Pleth->EEG, and EEG->Pleth for mice;

FIG. 8 shows the mean (points) and standard deviation (bars) of GPDC values per frequency band for ECG->EEG and EEG->ECG for humans;

FIG. 9 shows example ECG, breathing, and LDF perfusion signals recorded from a young, control subject;

FIG. 10 shows the mean (points) and standard error (vertical bars) of PDC values for all paired interactions across frequency bands for control (top) and T1D (bottom) subjects;

FIG. 11 shows a comparison of the directionality of PDC values for all paired interactions across frequency bands for control (top) and T1 D (bottom) subjects;

FIG. 12 shows the mean (points) and standard error (vertical bars) of GPDC values for all paired interactions across frequency bands for control (top) and T1 D (bottom) subjects; and

FIG. 13 shows a comparison of the directionality of GPDC values for all paired interactions across frequency bands for control (top) and T1 D (bottom) subjects.

### DETAILED DESCRIPTION

### I. Definitions

In the context of the present disclosure, the singular forms “a,” “an” and “the” can also include the plural forms, unless the context clearly indicates otherwise.

As used herein, the terms “comprises” and/or “comprising” can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

As used herein, the terms “first,” “second,” etc. can describe various elements, but these elements should not be limited by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “neuro-cardio-respiratory system” can refer to a complex network of at least a portion of a patient's brain, at least a portion of a patient's heart, and/or at least a portion of the patient's lungs. In some instances, the neuro-cardio-respiratory system can include additional organs, like at least a portion of the patient's musculature.

As used herein, the term “recording device” can refer to a modality that can record time series data. The time series data can correspond to one or more physiological signals to and/or from, and/or related to, and/or within interconnected regions within a same organ of a network and/or one or more imaging signals related to activity of at least one organ of the network.

As used herein, the term “physiological signal”, also referred to as “biosignal”, can refer to any electrical or non-electrical signal in a patient that can be continually measured and monitored. Examples of physiological signals can include intracranial electroencephalogram (EEG) signals, non-invasive EEG signals, electrocardiogram (ECG) signals, non-invasive magnetoencephalographic (MEG) signals, heart rate, blood pressure (BP) signals, blood perfusion signals, respiratory carbon dioxide (CO₂) signals, peripheral capillary oxygen saturation (SpO₂) signals, microvascular perfusion signals, plethysmography (Pleth) signals, or the like.

As used herein, the term “imaging signal” can refer to any signal that includes a visual representation of at least a portion of an interior of a patient's body over time. At an instance, the image can be used to represent the activity of one or more organs in a network. Examples of imaging signals can include functional magnetic resonance imaging (fMRI) signals, positron emission tomography (PET) signals, single photon emission computed tomography (SPECT) signals, computed tomography (CT) perfusion imaging signals, functional photoacoustic microscopy (f PAM) signals, magnetic particle imaging (MPI) signals, optical imaging signals, or the like.

As used herein, the term “network” can refer to a complex set of network interactions between different entities. The entities can include one or more organs and connections therebetween (e.g., neurological connections).

As used herein, the term “network interactions” can refer to bidirectional relations between different entities (e.g., organs) of the network.

As used herein, the term “organ” can refer to at least a portion of a self-contained biological structure (e.g., a patient's brain, heart, lungs, muscle, vasculature, etc.). For example, while the organ can include a patient's entire brain, the organ can also include the left hemisphere of the brain, the right hemisphere of the brain, or one or more portions of the left hemisphere and/or the right hemisphere. The same is true for the heart, lungs, muscle, vasculature, etc.

As used herein, the term “crisis” can refer to a sudden paroxysmal intensification of symptoms related to a dynamical disorder of one or more organs of the network. The dynamical disorder can be an epileptic seizure, conditions of status epilepticus (SE), sudden infant death syndrome (SIDS), sudden unexpected death in epilepsy (SUDEP), a myocardial infarction, a complication or crises related to diabetes, respiratory apnea, sleep disorders (like sleep apnea, restless legs syndrome, narcolepsy, insomnia, and the like), cardiac arrest, neurodegeneration related to Parkinson's disease, neurodegeneration related to Alzheimer's disease, essential tremor, stroke, respiratory attacks (e.g., asthma), acute respiratory failure, panic attacks, anxiety attacks, and sudden arrhythmic death syndrome.

As used herein, the term “abnormality” can refer to a state or quality of not being normal due to an anomaly, a deformity, a malformation, an impairment, a functional dysfunction, a deviation, or the like.

As used herein, the term “pathology” can refer to a symptom or cause that can be a biomarker for a crisis, an abnormality, and/or a treatment indicator. In some instances, the pathology can be a functional and/or structural neuronal damage in an afferent and/or an efferent connection between at least two organs and/or within at least one organ within a network. In other instances, the pathology can be and/or can be caused by Type 1 diabetes, Type 2 diabetes, a cardiac arrhythmia, ventricular tachycardia, apnea, ineffective sigh reflex, traumatic brain injury, neurodegeneration, stroke, epilepsy or the like.

As used herein, the term “estimation method” can refer to a method of mathematical analysis that can be used to estimate directional interactions between two or more organs in a network. The estimation method can be, for example, Coherence, Directed Coherence (DC), Partial Directed Coherence (PDC), Generalized Partial Directed Coherence (GPDC), Directed Transfer Function (DTF), Transfer Entropy, directed Mutual Information, Cross-Correlation, Dynamical Entrainment, Non-Linear Interdependence, Phase Synchronization, Phase Amplitude Coupling, Bispectrum, Bicoherence, measures of Centrality, or the like.

As used herein, the terms “treatment regime” and “treatment regimen” can refer to medical care given to a patient in response to detection of a crisis and/or an abnormality.

As used herein, the term “subject” can refer to any warm-blooded organism including, but not limited to, a human being, a pig, a rat, a mouse, a dog, a cat, a goat, a sheep, a horse, a monkey, an ape, a rabbit, a cow, etc. The terms “subject” and “patient” can be used interchangeably herein.

### II. Overview

The present disclosure relates to systems and methods that can perform network analysis of a patient's neuro-cardio-respiratory system to detect an impending crisis, to diagnose an abnormality, and/or to provide informative feedback about a treatment regime. By performing the network analysis, the systems and methods can perform a directed network mathematical analysis on a plurality of simultaneous physiological signals from each of the organs in the system, modeled as part of a unified and directed network within the system. The physiological signals can be, for example, intracranial electroencephalogram (EEG) signals, non-invasive EEG signals, electrocardiogram (ECG) signals, non-invasive magnetoencephalographic (MEG) signals, heart rate, blood pressure (BP) signals, blood perfusion signals, respiratory carbon dioxide (CO₂) signals, peripheral capillary oxygen saturation (SpO₂) signals, microvascular perfusion signals, plethysmography (Pleth) signals, or the like. Based on the directed network mathematical analysis, the systems and methods can perform a quantitative assessment of pathology related to interactions within the neuro-cardio-respiratory system. The systems and methods can detect a biomarker of a neurodegenerative and/or acquired dynamical disorder and then, when the biomarker is detected and/or exceeds a threshold, output an advance warning of an impending crisis, output information related to a diagnosis of an abnormality, and/or output information providing informative feedback about a treatment regime.

The systems and methods described herein achieve several advantages over previous solutions. The advantages include, but are not limited to: 1) performing localization of a functional pathology in feedforward and feedback loops of network connections between one or more organs (e.g., between peripheral organs and central nervous system (sensory and control units) from the resting state), 2) enabling risk assessment of susceptibility to crises with a high level of accuracy, 3) evaluating a prescribed treatment by monitoring the location and spatial extent of the functional pathology over time, and 4) providing advanced warning of crises, thus potentially becoming part of the treatment itself by timely changing (increasing and/or decreasing) the medication or informing programmable intelligent devices (e.g., deep brain stimulators for epilepsy and Parkinson's, cardiac pacemakers, defibrillators, or the like) when to intervene.

### III. Systems

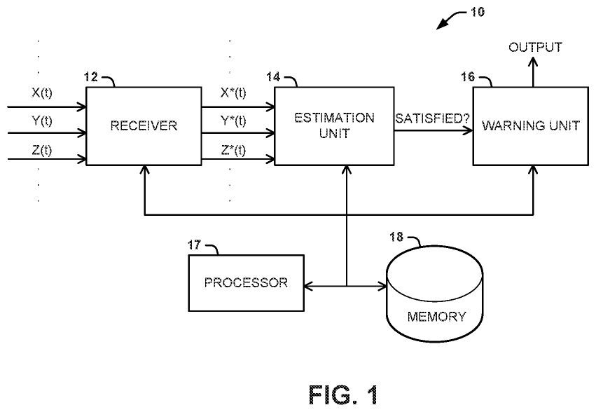

One aspect of the present disclosure can include a system 10 (shown in FIG. 1) that is configured to perform a network analysis of a patient's neuro-cardio-respiratory system, according to an aspect of the present disclosure. The system 10 can receive data (e.g., physiological signal(s) and/or imaging signal(s)) indicating directional communication between organs on a unified and directed network within the patient's neuro-cardio-vascular system. The system 10 can perform a directed network mathematical analysis on the data to perform a quantitative assessment of pathology related to interactions between the organs to detect an impending crisis, to diagnose an abnormality, and/or to provide informative feedback about a treatment regime based on the quantitative assessment of the pathology.

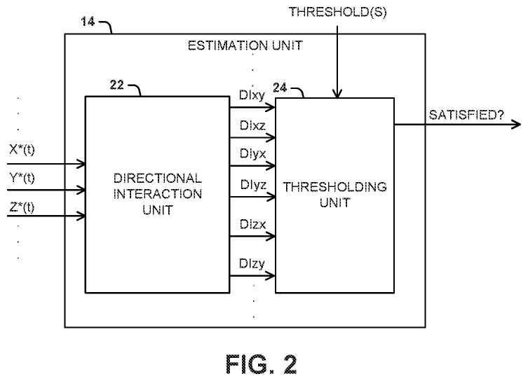

FIG. 1 and associated FIG. 2 are schematically illustrated as block diagrams with the different blocks representing different components. The functions of one or more of the components can be implemented by computer program instructions. These computer program instructions can be provided to a processor 17—for example, of a general purpose computer, special purpose computer, and/or other programmable data processing apparatus to produce a machine, such that the instructions, which execute via the processor 17, create a mechanism for implementing the functions of the components specified in the block diagrams.

These computer program instructions can also be stored in a non-transitory computer-readable memory 18 that can direct a computer or other programmable data processing apparatus to function in a particular manner, such that the instructions stored in the non-transitory computer-readable memory 18 (also referred to as a computer-readable storage medium) to produce an article of manufacture including instructions, which implement the function specified in the block diagrams and associated description.

The computer program instructions can also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions of the components specified in the block diagrams and the associated description.

Accordingly, the system 10 described herein can be embodied at least in part in hardware and/or in software (including firmware, resident software, micro-code, etc.). Furthermore, aspects of the system 10 can take the form of a computer program product on a computer-usable or computer-readable storage medium having computer-usable or computer-readable program code embodied in the medium for use by or in connection with an instruction execution system. A computer-usable or computer-readable medium can be any non-transitory medium that is not a transitory signal and can contain or store the program for use by or in connection with the instruction or execution of a system, apparatus, or device. The computer-usable or computer-readable medium can be, for example but not limited to, an electronic, magnetic, optical, electromagnetic, infrared, or semiconductor system, apparatus or device. More specific examples (a non-exhaustive list) of the computer-readable medium can include the following: a portable computer diskette; a random access memory; a read-only memory; an erasable programmable read-only memory (or Flash memory); and a portable compact disc read-only memory.

The system 10 can include components including at least a receiver 12, an estimation unit 14, and a warning unit 16. One or more of the components can include instructions that are stored in a non-transitory memory 18 and executed by a processor 17. Each of the components can be in a communicative relationship with one or more of the other components, the processor 17, and/or the non-transitory memory 18 (e.g., via a direct or indirect electrical, electromagnetic, optical, or other type of wired or wireless communication) such that an action from the respective component causes an effect on one or more of the other components.

The receiver 12 can be configured to receive data recorded by one or more recording devices from a patient (the recording devices can be physical hardware, such as including at least one sensor, transducer, or the like). The data can time varying and related to two or more organs of a neuro-cardio-respiratory system (e.g., a patient's brain, heart, lungs, muscle, vasculature, etc.) within the patient's body that are monitored. The data can include physiological signal(s) and/or imaging signal(s) that are time varying. As shown in FIG. 1, the data can be referred to as X(t), Y(t), and Z(t). It will be understood, however, the data can include a greater number of time varying signals or a lesser number of time varying signals.

The time series data (X(t), Y(t), Z(t)) received as inputs by the receiver 12 can include raw time series signals obtained from or generated by the one or more recording devices. It will be understood that the time series data (X(t), Y(t), Z(t)) can each include one or more signals recorded by from one or more recording channels of the one or more recording devices. For example, X(t) can include EEG signals from 100 EEG channels. In some instances, the receiver 12 can preprocess one or more of the raw time series signals into preprocessed time series signals (X*(t), Y*(t), Z*(t), for example). Similarly, the preprocessed time series data (X*(t), Y*(t), Z*(t)) can each include the same or processed to be greater or smaller one or more signals recorded by from one or more recording channels of the one or more recording devices. The term “preprocessed time series signals (X*(t), Y*(t), Z*(t))” can refer to input to the estimation unit 14 to prevent confusion with the time series data (X(t), Y(t), Z(t)) that is input to the receiver 12.

The receiver 12 can provide the time series data (X(t), Y(t), Z(t)) and/or the preprocessed data (X*(t), Y*(t), Z*(t)) to the estimation unit 14. For example, the receiver 12 can divide the time series data (X(t)) into a series of time epochs so that the estimation unit 14 can perform its analysis for the different epochs (e.g., each epoch corresponds to a time period). The epochs can be non-overlapping or random (e.g., containing one or more overlapping portions). For simplicity of illustration and explanation, the receiver 12 is illustrated as providing the preprocessed time series signals (X*(t), Y*(t), Z*(t)) to the estimation unit 14. Although the preprocessed time series signals (X*(t), Y*(t), Z*(t)) is referenced herein, it will be appreciated that the receiver 12 need not perform the preprocessing step and can provide the time series signals (X(t), Y(t), Z(t)) to the estimation unit 14. The preprocessed time series signals (X*(t), Y*(t), Z*(t)) possesses vector properties similar to those as defined for the time series signals (X(t), Y(t), Z(t)).

The estimation unit 14 can be configured to estimate directional interactions between the two or more organs within the patient's body over time via a mathematical analysis of the data (e.g., the time series signals and/or the preprocessed time series signals) to identify one or more pathologies in a network of the neuro-cardio-respiratory system. The estimate of the directional interactions can include determining an information inflow towards each of the two or more organs based on an analysis of the data (conducted using at least one estimation method, for example: Coherence, Directed Coherence (DC), Partial Directed Coherence (PDC), Generalized Partial Directed Coherence (GPDC), Directed Transfer Function (DTF), Transfer Entropy, Mutual Information, Correlation Dimension, Dynamical Entrainment, Non-Linear Interdependence, Phase Synchronization, Phase Amplitude Coupling, Bispectrum, Bicoherence, etc.). The information inflow can reflect a directional flow of information to each of the at least two organs from at least one other organ.

The one or more pathologies can be identified based on the estimate of directional interactions. For example, as shown in FIG. 2, the estimation unit 14 includes a directional interaction unit 22, which can take as input the time series signals and/or the preprocessed time series signals (X*(t), Y*(t), Z*(t)) and output directional interactions (Dlxy, Dlxz, Dlyx, Dlyz, Dlzx, Dlzy). The directional interactions (Dlxy, Dlxz, Dlyx, Dlyz, Dlzx, Dlzy) can be biomarkers for certain impending crises and/or abnormality and/or effectiveness/lack of effectiveness of a treatment regime. As an example, the directional interaction unit 22 can fit a multidimensional model (e.g., a vector autoregressive model (VAR) or a multivariate auto-regressive model (MVAR)) of order p (e.g., a pre-determined value or an optimally determined value) to successive segments of the time series signals and/or the preprocessed time series signals (X*(t), Y*(t), Z*(t)); and determine (using Directed Coherence (DC), Partial Directed Coherence (PDC), Generalized Partial Directed Coherence (GPDC), or Directed Transfer Function (DTF)) a measure of inflow from each of the at least two organs to at least one other organ in a plurality of frequency bands. For example, the plurality of frequency bands can be analyzed for all signal ranges from the minimum sampling rate to the maximum available frequency according to the Nyquist theorem (B/2) (where B is a frequency at which the signal was sampled).

An example using X*(t) and GPDC will be described. In this example, an multivariate autoregressive model MVAR(p) can be constructed of an order p (where p is a pre-determined value—like an autocorrelation of the time series data—or an optimally determined value). For example, an autoregressive model can be expressed as:

$$X ( t ) = {\sum }_{τ = 1}^{p} A ( τ ) X ( t - τ ) + E ( t ) \qquad Equation 1$$

where p is the model order, and τ is the time delay in data points. All of the model coefficients are coupled with an error term E, which is representative of the error of the model. Taking the discrete Fourier Transform of X(t):

$$B ( f ) = I - {\sum }_{τ = 1}^{p} A ( τ ) {e}^{- i 2π f τ} \qquad Equation 2$$

Where the new coefficients are subtracted from the identity matrix to eliminate the inflow of channels to themselves. Finally, employing spectral factorization on B(f), GPDC is defined according to Equation 3. GPDC provides a measure for the direct linear influence of organ Xⱼ on organ Xᵢ at frequency f conditioned by the rest of the signal variables:

$$GPDCij ( f ) = \frac{\frac{\vert Bij ( f ) \vert}{σ ij}}{\sqrt{\frac{{\vert Bijk ( f ) \vert}^{2}}{{σ}^{2} kk}}} , \qquad Equation 3$$

where αᵢⱼ is obtained from the covariance matrix of ε(t), S=[α_{ιj}]_{i, j=1-n}, and Bᵢⱼ(f) is the (i,j)^{th} element of the matrix

$$B ( f ) = I - {\sum }_{τ - 1}^{p} B ( τ ) {e}^{- i 2π f τ} ,$$

where I is the n×n identity matrix.

The average directional connectivity index between nodes (corresponding to information flows for organs on the network) based on the quantification of the network connectivity (e.g., from the GPDC) over a given frequency range (f₁, f₂) Hz. The information inflow to a particular organ can be determined by a weighted sum of the information inflows from the rest of the nodes j. For example, assuming a simple sum:

$$InDi = {\sum }_{j = 1 , j \neq i}^{n} ( GPDCj -> i ( f ) ) . \qquad Equation 4$$

The directional interactions (Dlxy, Dlxz, Dlyx, Dlyz, Dlzx, Dlzy) can be input to a thresholding unit 24, which can compare each of the directional interactions (Dlxy, Dlxz, Dlyx, Dlyz, Dlzx, Dlzy) to a threshold (or thresholds) (THRESHOLD(s)) to determine whether one or more conditions are satisfied that indicate a warning is necessary (SATISFIED?). The threshold (or thresholds) (THRESHOLD(s)) can be predetermined values related to specific interactions that can be stored either locally or remotely. As an example, one or more thresholds can be determined from data from normal subjects (e.g., experimental data), data from previous patients with similar characteristics (e.g., same disease state, same age, same sex, etc.), from previous data related to the patient, or the like. For example, Dlxy can be compared to a threshold for Dlxy; if Dlxy is greater than a threshold for Dlxy, the thresholding unit 24 can signal the warning unit 16 of FIG. 1 to provide an output (OUTPUT) notifying of the exceeded threshold.

The warning unit 16 can be configured to provide an output (OUTPUT) when conditions are satisfied (SATISFIED?) related to the identification of the one or more pathologies in the network of the neuro-cardio-respiratory system. The output (OUTPUT) can be an advance warning of the impending crisis, a diagnosis of an abnormality, or the like. A treatment regime may be prescribed in response to the advance warning or the diagnosis. Any subsequent output (OUTPUT) by the warning unit can include informative feedback for the prescribed treatment regime by continuing to monitor the patient during and after treatment.

The output (OUTPUT) can be an alarm or other notification related to the patient. For example, the output (OUTPUT) can be an auditory, visual, tactile, etc., notification at the patient's bedside. Also, the output (OUTPUT) can be an auditory, visual, tactile, etc., notification sent to associated medical professionals (e.g., doctors, nurses, etc.). As an example, the output (OUTPUT) can be an issued code related to the patient requiring medical action. As a further example, the output (OUTPUT) can be noted in the patient's chart, a treatment regime can be prescribed, and the patient tracked over time based on a prescribed treatment regime.

### IV. Methods

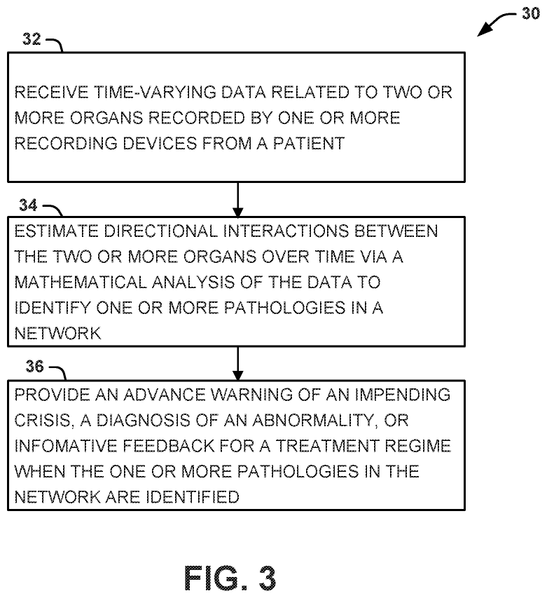

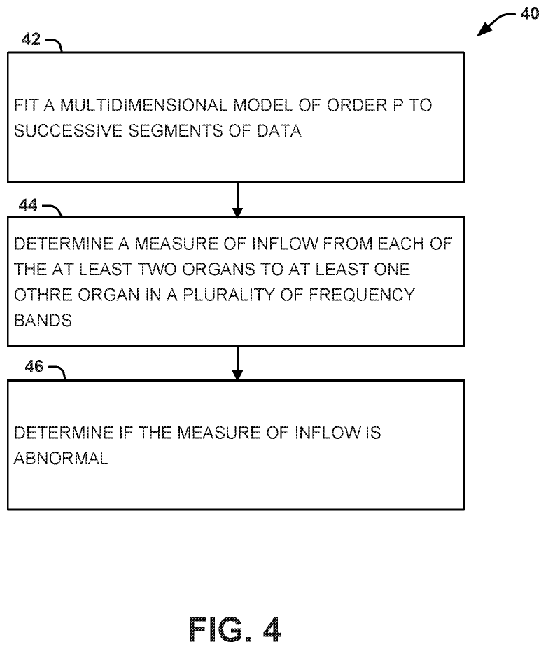

Another aspect of the present disclosure can include methods for performing a network analysis of a patient's neuro-cardio-respiratory system. FIG. 3 is an example of a method 30 for performing a network analysis of a patient's neuro-cardio-respiratory system. FIG. 4 is an example method for estimating directional interactions between two or more organs. The methods 30, 40 may be performed using the components of the system 10, for example.

The methods 30 and 40 of FIGS. 3 and 4, respectively, are illustrated as process flow diagrams with flowchart illustrations. For purposes of simplicity, the methods 30 and 40 are shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the methods 30 and 40.

One or more blocks of the respective flowchart illustrations, and combinations of blocks in the flowchart illustrations, can be implemented by computer program instructions. These computer program instructions can be stored in memory and provided to a processor of a general purpose computer, special purpose computer, and/or other programmable data processing apparatus to produce a machine, such that the instructions, which execute via the processor of the computer and/or other programmable data processing apparatus, create mechanisms for implementing the steps/acts specified in the flowchart blocks and/or the associated description. In other words, the steps/acts can be implemented by a system comprising a processor that can access the computer-executable instructions that are stored in a non-transitory memory.

The methods 30 and 40 of the present disclosure may be embodied in hardware and/or in software (including firmware, resident software, micro-code, etc.). Furthermore, aspects of the present disclosure may take the form of a computer program product on a computer-usable or computer-readable storage medium having computer-usable or computer-readable program code embodied in the medium for use by or in connection with an instruction execution system. A computer-usable or computer-readable medium may be any non-transitory medium that can contain or store the program for use by or in connection with the instruction or execution of a system, apparatus, or device.

As noted, FIG. 3 is an example of a method 30 for performing a network analysis of a patient's neuro-cardio-respiratory system. At 32, time-varying data from a patient related to one or more organs recorded by one or more recording devices can be received (e.g., by receiver 12). The time-varying data can be physiological signal(s) and/or imaging signal(s)) indicating directional communication between organs on a unified and directed network within the patient's neuro-cardio-vascular system. At 34, an estimate of directional interactions between the two or more organs over time can be made (e.g., by estimation unit 14) via a mathematical analysis of the data to identify one or more pathologies in a network. This quantitative assessment of pathology related to interactions between the organs to detect an impending crisis, to diagnose an abnormality, and/or to provide informative feedback about a treatment regime based on the quantitative assessment of the pathology. At 36, an alarm can be provided (e.g., by warning unit 16) in response to the quantitative assessment. The alarm can provide an advance warning of an impending crisis a diagnosis of an abnormality, and/or informative feedback for a treatment regime when the one or more pathologies are identified. As an example, the impending crisis (an oncoming seizure) can be identified based on impaired interaction between one or more organs or within portions of one or more organs (see U.S. Pat. No. 9,730,628 B1, entitled System and Method for Identifying a Focal Area of Abnormal Network Interactions in the Brain, which is incorporated by reference herein in its entirety). In U.S. Pat. No. 9,730,628 B1, the pathological region was identified as the region that most frequently receives maximum inflow with respect to other brain regions (and is thus maximally controlled) during the seizure-free (interictal) periods. In this case, the threshold is not a number, but a maximal value of a plurality of interactions.

FIG. 4 is an example method for estimating directional interactions between two or more organs (e.g., performed by the estimation unit 14). At 42, a multidimensional model of order p can be fit to successive segments of data. At 44, a measure of inflow can be determined from each of the at least two organs to at least one other organ in a plurality of frequency bands. At 46, it can be determined whether the measure of inflow is abnormal.

### V. Example

The following examples are for the purpose of illustration only and are not intended to limit the scope of the appended claims.

### Epilepsy

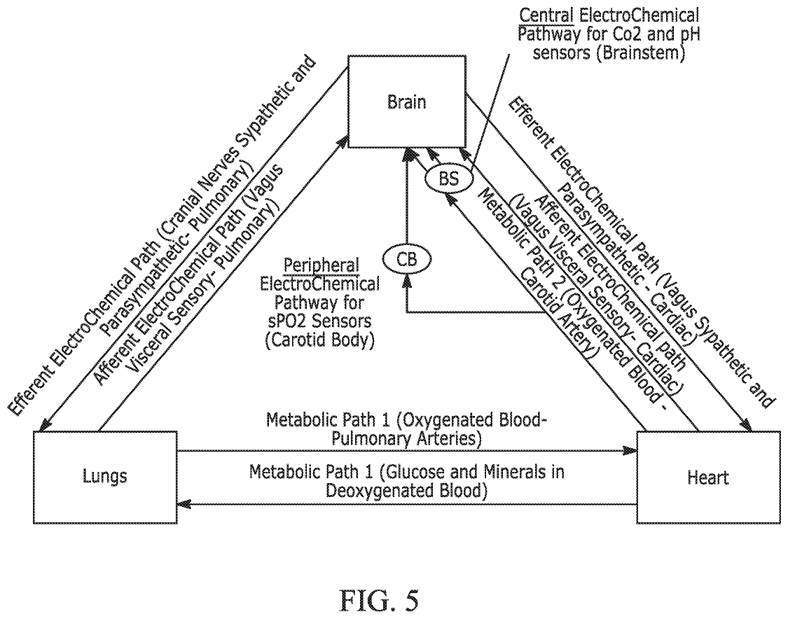

This example shows network analysis of the neuro-cardio-respiratory system (brain-heart-lung interactions are schematically shown in FIG. 5) in epilepsy done on data collected from Kcna1-knockout mice and epileptic humans.

### Methods

### Mice

Kcna1-knockout mice (in other words, mice lacking the Kcna1 gene) exhibit neuronal hyper-excitability and are SUDEP-prone. At Louisiana State University, Health Sciences Center—Shreveport, Shreveport, La., Kcna1-knockout (n=8) and wild-type (n=7) mice of both sexes were anesthetized and surgically implanted with 6 bilateral EEG electrodes (4 recording channels, 1 reference, 1 ground) overlying left and right temporal and parietal cortex and two frontal cortex (reference and ground) electrodes. Two thoracic electrodes were tunneled subcutaneously on either side to record an ECG. Mice were allowed to recover for 1 day before recording 8 h of continuous EEG-ECG-Pleth in an unrestrained whole-body plethysmography chamber. EEG, ECG and Pleth were sampled at 1 kHz, 2 kHz and 500 Hz respectively. EEG and ECG were subsequently down-sampled to 500 Hz. The first 4 h (mostly seizure-free) of the recordings were analyzed.

### Humans

Intracranial EEG and ECG were continually recorded in 35 patients undergoing phase II monitoring in the epilepsy monitoring unit (EMU) (all data provided by the University of Alabama at Birmingham, Neurology Department, Birmingham, Ala. During this time, the patients had their anti-epileptic drugs (AEDs) reduced, and had 2-3 seizures of the same kind before being released. The data for the full record (3-10 days) was used—EEG typically has 100-250 channel, sampled at 500 Hz or 2 kHZ, ECG has two or single channel, and is sampled at the same rate as EEG.

### Directional Interactions

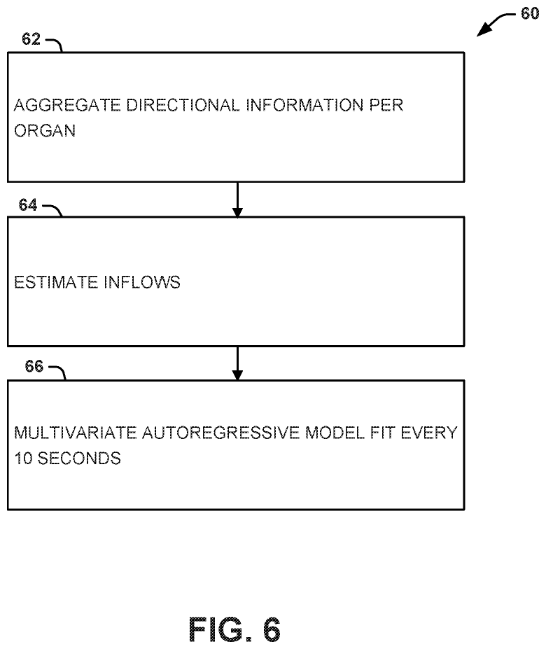

An example of the process 60 for determining directional interactions is shown in FIG. 6, where directional information is aggregated per organ (Step 62), inflows are estimated (Step 64), and a multivariate autoregressive model is fit every 10 seconds (Step 66) Directional interactions with respect to information flow between brain, heart and lungs were estimated by fitting a 5- or 6-dimensional/channel (3-4 EEG sites, 1 ECG site, 1 Pleth signal) multivariate autoregressive (MVAR) model of order 7 that was fit to short (10 s) consecutive and non-overlapping EEG, ECG, and Pleth data segments, and the Generalized Partial Directed Coherence (GPDC) connectivity measure (inflow) values were estimated over 4 hours. The GPDC inflow values were estimated over 13 frequency bands, from 1 to 500 Hz (mouse) or 1 to 110 Hz (human) and were then aggregated per interaction and frequency band for each genotype. The GPDC inflow values were then aggregated per organ (e.g., sum of GPDC values from the 4 (mice) or 22 (human) different brain sites to the heart and four (mice) or 22 (human) different GPDC values from the heart to the brain) and frequency band. However, results from a single human are described. In this human, during a recording session during which the patient died from cardiac arrest. 3.7 hours prior to the onset of SE (following a clinical seizure), the brain-heart interactions until SE were measured (brain interactions reflected in an EEG measured by scalp electrodes). Additionally, similar results from a human with brain interactions reflected in an EEG measured invasively are shown in Hutson T, Pizarro D. Pati S and lasemidis LD (2018) Predictability and Resetting in a Case of Convulsive Status Epilepticus. Front. Neurol. 9:172, which is incorporated by reference in its entirety.

### Results

### Mice

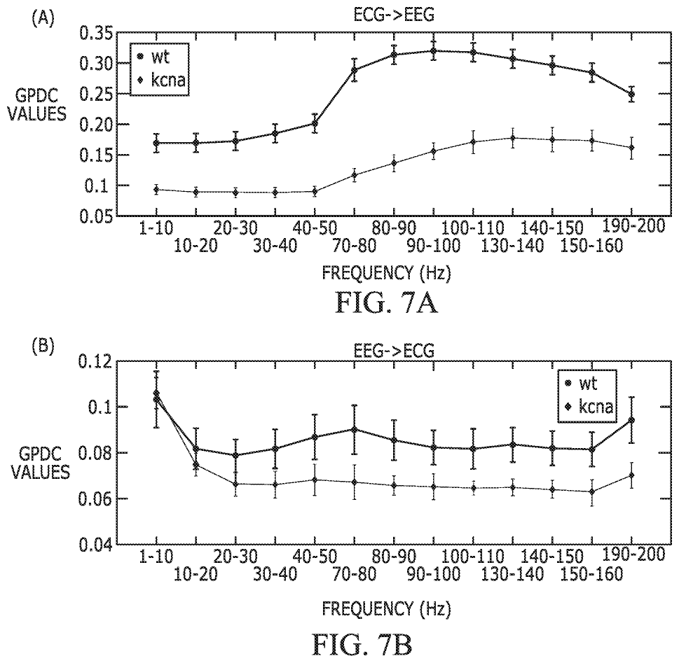

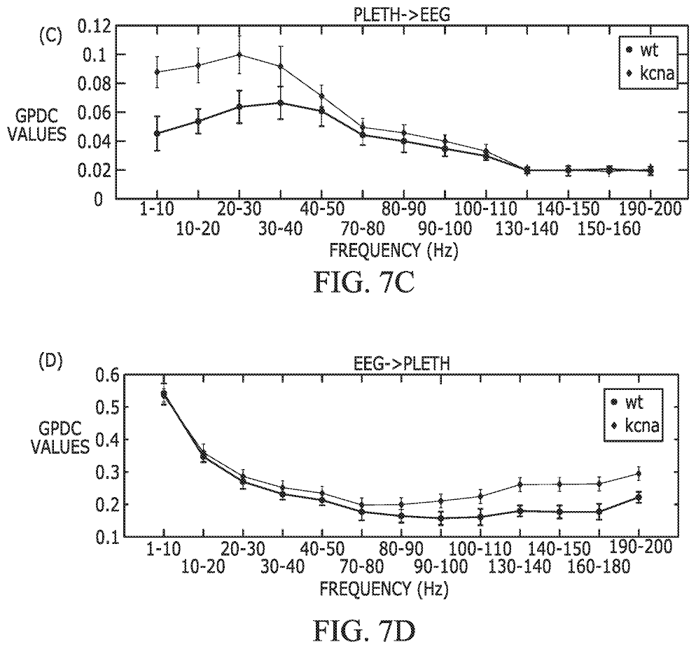

The mean and standard error of the mean of GPDC inflow values per frequency band for heart<->brain and lung<->brain interactions are shown for the two genotypes (wild type—wt and kcna1) in FIG. 7 (heart<->brain, elements a and b, lungs<->brain, elements c and d). Three statistically significant observations (p<0.001) were made: 1) SUDEP-prone mice (kcna1) exhibit a reduced functional connection in heart-brain interactions across frequencies (FIGS. 7 (a) and (b)); 2) In both healthy (wt) and SUDEP-prone (kcna1) mice, the connectivity of lung<->brain interactions is reduced in high frequencies (FIGS. 7 (c) and (d)); 3) Relative to wild-type (wt), SUDEP-prone (kcna1) mice exhibit both an increased lung->brain feedback in lower frequencies and an increased brain->lung connectivity in higher frequencies (FIG. 7 (d)).

Heart<->brain interactions were impaired in both directions in SUDEP-prone mice (kcna1) when the interactions of the heart and brain with the lungs are also taken in consideration. This impairment may lead to abnormally high lungs<->brain interactions in SUDEP-prone (kcna1) mice, possibly as a compensatory mechanism of the pathology.

### Humans

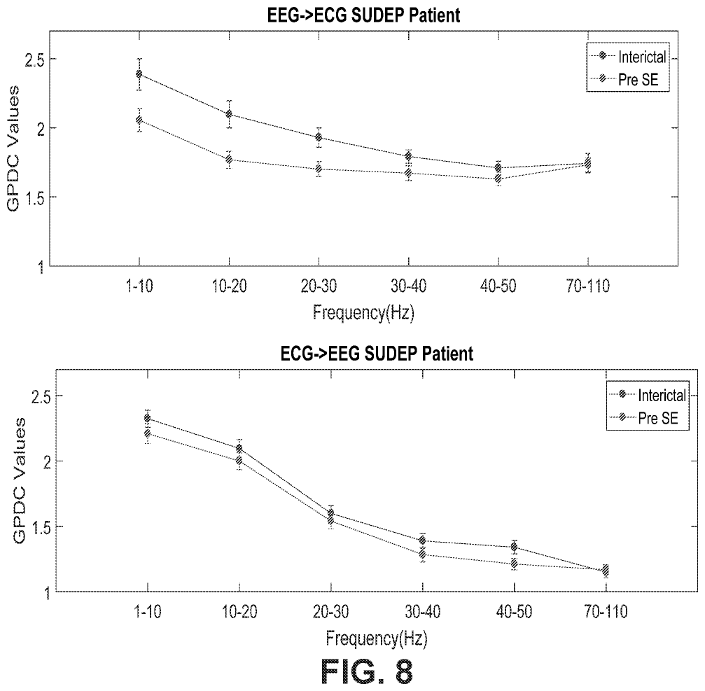

The mean and standard deviation from the mean of the GPDC values per frequency band from ECG to EEG (element a) and from EEG to ECG (element b) for two selected periods of the recording denoted as interictal (˜19.5 hrs) and Pre-Status Epilepticus (pre-SE) (˜3.7 hours) are shown in FIG. 8. From these results, two statistically significant observations are made: 1) the ECG->EEG information flow in the pre-SE period is reduced compared to the interictal period, and 2) the EEG->ECG information flow in the pre-SE period is more significantly reduced compared to the interictal period. That is, across the 3.7 hours pre-SE period, the EEG->ECG interactions were significantly lower (impaired) than the rest of the time (˜19.5 hrs) long before SE onset. This suggests that the impending crisis of SE can be identified by the measures described herein. This conjecture is further supported from the dynamics of the measures of the directional interactions during the transition to SE, as shown in Hutson T, Pizarro D, Pati S and lasemidis LD (2018) Predictability and Resetting in a Case of Convulsive Status Epilepticus. Front. Neurol. 9:172, which is incorporated by reference in its entirety. The patient experienced a severe, but non-fatal, case of SE and, about 3 to 4 hours before SE onset, abnormal directional network interactions in the brain were discovered that could have been used as a predictive tool for the onset of SE and also show the recovery of the network interactions during medical intervention, thus suggesting the use of the method described herein as an evaluation of pathology treatment too.

Notably, higher frequencies in the EEG and ECG (150-160 Hz) showed more significant predictive trends between the heart focal sites of a seizure in the brain than between the heart and focal sites of a seizure in the brain in lower frequencies (1-10 Hz). Accordingly, higher frequency bands of electrocardiogram (ECG) signals (greater than a traditional 0 to 2 Hz band, such as greater than 80 Hz) are given higher weight in quantification of the directed interactions between the brain and the heart.

### Type 1 Diabetes

This example shows network analysis of the neuro-cardio-respiratory system in Type 1 Diabetes (T1D). In this instance, the neuro-cardio-respiratory system is an oscillatory system that includes the cardiac system, the respiratory system, and the vasomotor system.

### Methods

Two groups of 10 healthy controls (age: 26.7±1.5 years; M/F: 7/3) and 10 T1 D patients (age: 29.7±13.3 years; M/F: 5/5) were recruited for this study (data provided by the Department of Information Engineering, University of Florence, Florence, Italy). 1 control (i.e., ≈10%) and 4 T1 D (i.e., ≈40%) subjects were smokers. Information regarding T1 D-related complications, time since diagnosis and HbA1c levels (namely, the latest recorded value and the annual average) were retrieved for each diabetic subject.

Measurement sessions involved the simultaneous acquisition of electrocardiogram (ECG), breathing, and microvascular perfusion signals. Microvascular Perfusion was measured on the distal phalanx of the right forefinger using a Periflux 5000 laser Doppler flowmetry (LDF) system (Perimed, Sweden). The time constant of the output low-pass filter of the instrument was set to 0.03 s. The heart and spontaneous respiratory activities were monitored by means of a BioHarness 3.0 wearable chest strap sensor (Zephyr Technology, US), and transmitted to a PC via Bluetooth. All the mean breathing rates of the evaluated subjects were inside the nominal physiological range, i.e., (0.145, 0.6) Hz.

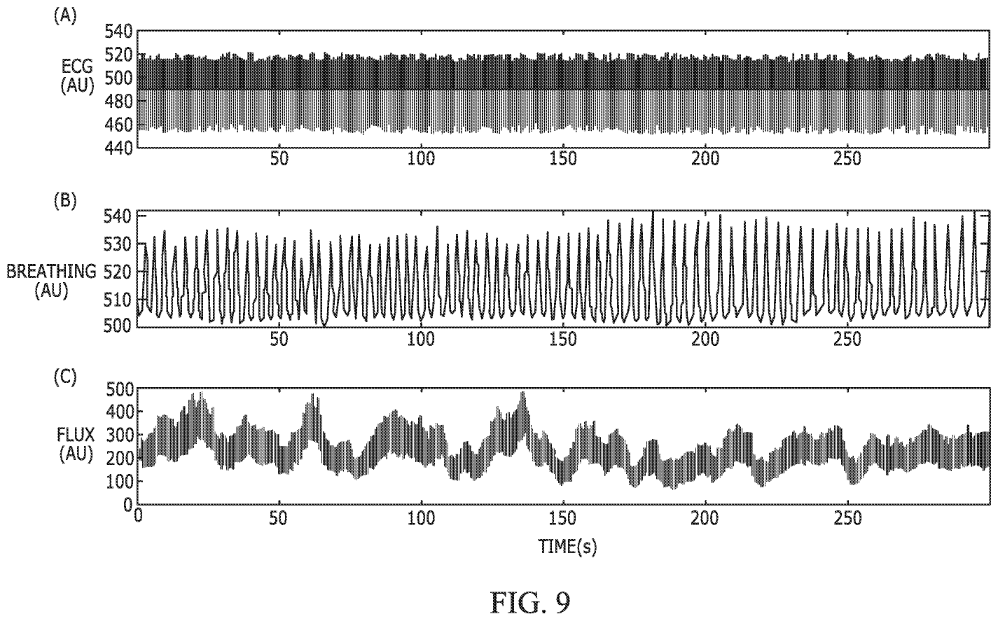

The three signals (FIG. 9, elements (a)—cardiac from a control individual, (b)—respiratory from a control individual, and (c)—myogenic from a control individual) were digitized at a sampling frequency of 250 Hz and synchronized through a customized data acquisition software. Measurements lasted 5 min and took place in thermally stable conditions (T≈23° C.), following a preliminary acclimatization time interval of ≈10 min. During signal acquisition, subjects were seated in a chair with back support and leaned their right forearm on a table; furthermore, the subjects were instructed to carefully avoid abrupt movements to prevent the displacement of the LDF probe and thus the introduction of motion-related artifacts in the perfusion data.

### Results

Estimation of partial directed coherence (PDC) and generalized partial directed coherence (GPDC) was performed using the T-Mullen-Sift Toolbox for MATLAB [http://www.antillipsi.net/research/software]. A model order of 7 was used to fit 10 s consecutive segments of ECG, Respiratory, and LDF data via the Viera-Morf optimization algorithm. PDC and GPDC were then estimated for each subject on the resulting 3-channel model for the full duration of the record and across the frequency spectrum from 0.052-2 Hz described previously in dynamical Baysian interference (DBI) methodology. These values for each of the 6 directional interactions were combined across subjects and time for each group (healthy, T1 D) and frequency band.

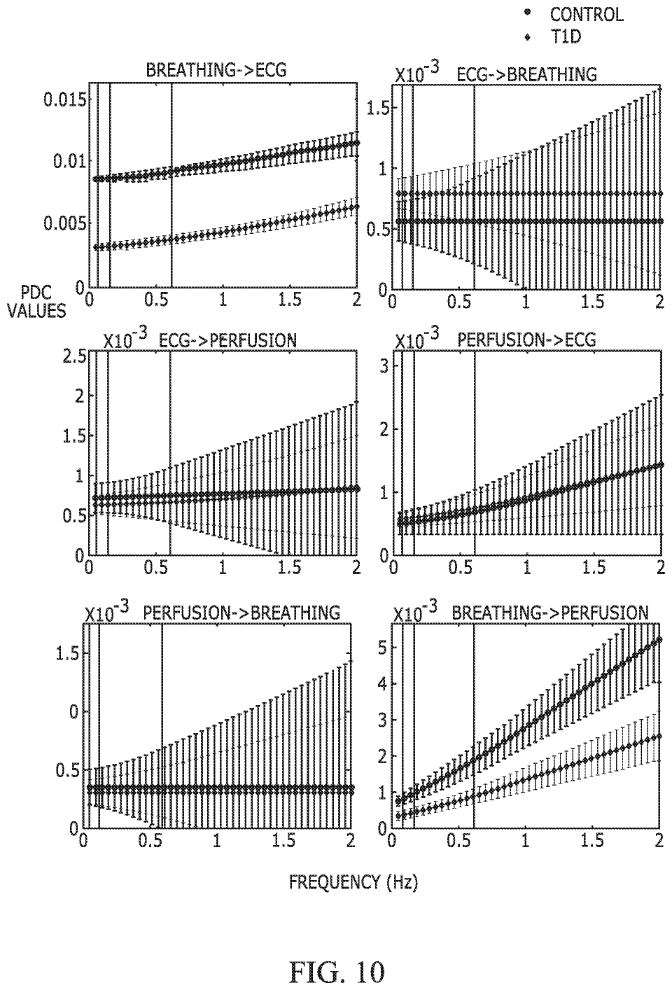

In FIG. 10, the means (points) and standard errors (vertical bars) of PDC values for each interaction and frequency are shown. From these connectivity values, the following statistically significant observations can be made: Breathing->ECG and Breathing->Perfusion interactions are impaired across frequencies for T1D subjects. Other interactions do not exhibit statistically significant differences between groups. In general for PDC values, variance of connectivity increases with frequency.

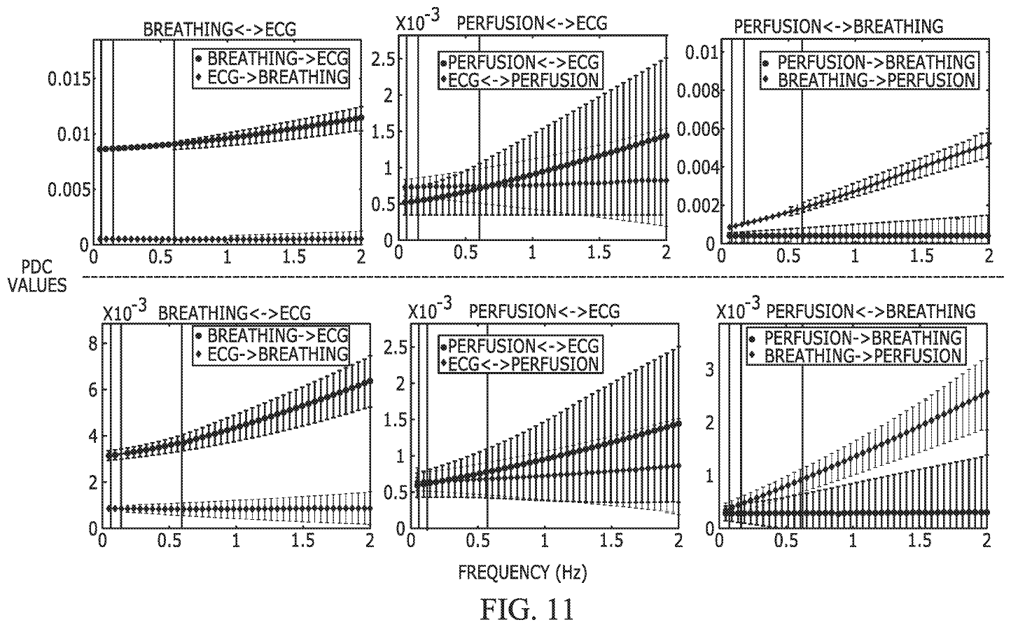

Directionality of paired interactions is compared across frequencies for control (top) and T1 D (bottom) subjects, as shown in FIG. 11. For both groups, Breathing->ECG exhibits much higher connectivity values than ECG->Breathing across frequencies. In control subjects, ECG->Perfusion is lower than Perfusion->ECG below ˜0.8 Hz (entering into the range of traditional cardiac frequencies) above which Perfusion->ECG is higher, suggesting potential high-frequency feedback from vasomotor oscillatory function to cardiac activity. For T1 D subjects, both directions are similar in magnitude until ˜0.5 Hz, above which Perfusion->ECG is again higher than ECG->Perfusion. Breathing->Perfusion (right most plots) is higher in connectivity values than Perfusion->Breathing for both groups across frequencies. T1 D subjects exhibit higher variance than control subjects for both directions of the Perfusion<->Breathing interaction, suggesting higher instability of interactions across subjects and over time.

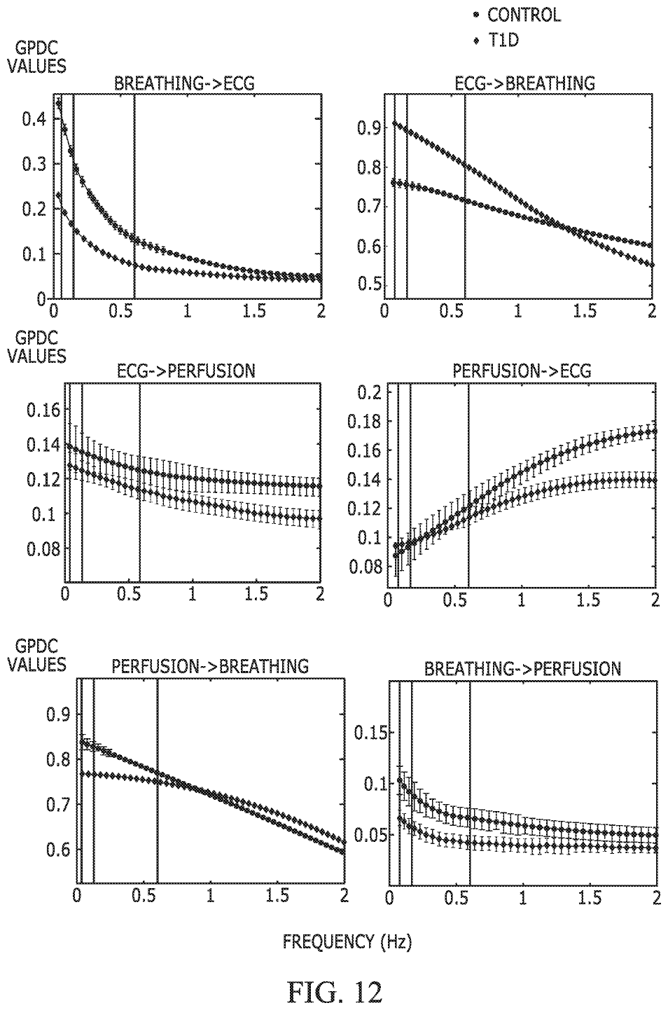

GPDC was also estimated across the data, the means and standard errors for which are plotted in FIG. 12. Several statistically significant observations are made. As in the PDC estimations, Breathing->ECG is impaired across frequency for T1D subjects. The strength of this interaction decreases with frequency in GPDC as opposed to the increase in magnitude across frequency elucidated through PDC. Unlike PDC, GPDC values reveal abnormally high ECG->Breathing interactions for T1 D subjects below 1.3 Hz potentially acting as a compensatory mechanism for the impairment from Breathing->ECG. Above 1.5 Hz (in the upper region of traditional cardiac activity frequency range) T1 D subjects exhibit lower magnitudes of coupling for ECG->Breathing. GPDC values for both directions of ECG<->Perfusion interactions above 0.5 Hz (in the cardiac nominal frequency range) are impaired in T1 D subjects. Perfusion->Breathing connectivity is impaired in T1 D subjects at frequencies below 0.5 Hz and Breathing->Perfusion values are impaired in T1 D subjects across frequencies.

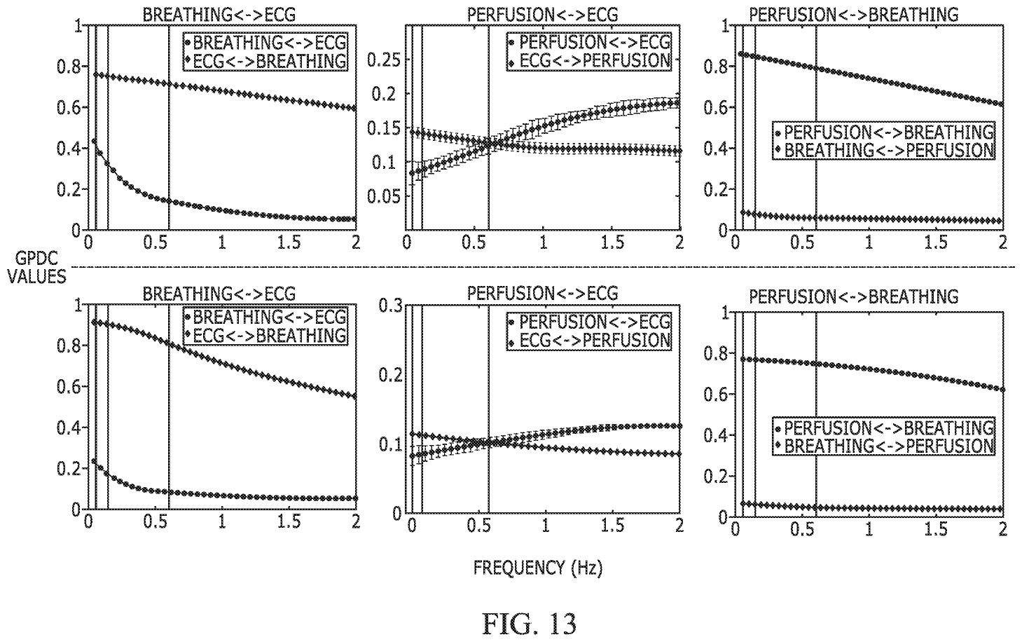

Directionality of GPDC values for all paired interactions is compared across frequencies for control (top) and T1 D (bottom) subjects in FIG. 13. Opposite of PDC, GPDC exhibits higher ECG->Breathing than Breathing->ECG connectivity for both groups. ECG->Perfusion is the stronger than Perfusion->ECG at frequencies under 0.5 Hz, over which the directionality of this interaction swaps for both T1 D and control subjects. Across frequencies and for both T1 D and control groups, Perfusion->Breathing exhibits higher connectivity values than Breathing->Perfusion, which is also opposite of the directionality found in PDC.

From the above description, those skilled in the art will perceive improvements, changes and modifications. Such improvements, changes and modifications are within the skill of one in the art and are intended to be covered by the appended claims.

What is claimed is:

1. A method comprising:
receiving, by a system comprising a processor, data recorded by one or more recording devices from a patient, wherein the data is time varying and related to two or more organs of a neuro-cardio-respiratory system within the patient's body that are monitored;
estimating, by the system, directional interactions between the two or more organs within the patient's body over time via a mathematical analysis of the data to identify one or more pathologies in a network of the neuro-cardio-respiratory system; and
providing, by the system, an advance warning of an impending crisis, a diagnosis of an abnormality, or informative feedback for a treatment regime when the one or more pathologies in the network of the neuro-cardio-respiratory system are identified.

2. The method of claim 1, wherein the data comprises one or more physiological signals and/or one or more images related to activity of at least one of the two or more organs.

3. The method of claim 1, wherein the two or more organs include at least two of the patient's brain, the patient's heart, and the patient's lungs.

4. The method of claim 3, wherein the data comprises one or more physiological signals related to the patient's brain, the patient's heart, and/or the patient's lungs,
wherein the one or more physiological signals are related to one or more of intracranial electroencephalogram (EEG) signals, non-invasive EEG signals, electrocardiogram (ECG) signals, non-invasive magnetoencephalographic (MEG) signals, heart rate, blood pressure (BP) signals, blood perfusion signals, respiratory carbon dioxide (CO₂) signals, peripheral capillary oxygen saturation (SpO₂) signals, microvascular perfusion signals, and plethysmography (Pleth) signals.

5. The method of claim 3, wherein the data comprises one or more imaging signals related to the patient's brain, the patient's heart, and/or the patient's lungs,
wherein the one or more imaging signals comprise one or more of functional magnetic resonance imaging (fMRI) signals, positron emissions tomography (PET) signals, single photon emission computed tomography (SPECT) signals, computed tomography (CT) perfusion imaging signals, functional photoacoustic microscopy (fPAM) signals, magnetic particle imaging (MPI) signals, and optical imaging signals.

6. The method of claim 1, wherein the estimating further comprises:
determining an information inflow towards each of the two or more organs based on the mathematical analysis of the data,
wherein the information inflow reflects a directional flow of information to each of the at least two organs from at least one other organ.

7. The method of claim 6, wherein the determining the information inflow is conducted using at least one estimation method,
wherein the at least one estimation method comprises at least one of Coherence, Directed Coherence (DC), Partial Directed Coherence (PDC), Generalized Partial Directed Coherence (GPDC), Directed Transfer Function (DTF), Transfer Entropy, Mutual Information, Cross Correlation, Dynamical Entrainment, directed measures of Centrality, Non-Linear Interdependence, Phase Synchronization, Phase Amplitude Coupling, Bispectrum, and Bicoherence.

8. The method of claim 6, further comprising:
fitting a multidimensional model of order p to successive segments of the data; and
determining a measure of inflow from each of the at least two organs to at least one other organ in a plurality of frequency bands,
wherein the determining is conducted using Directed Coherence (DC), Partial Directed Coherence (PDC), Generalized Partial Directed Coherence (GPDC), or Directed Transfer Function (DTF).

9. The method of claim 8, wherein the plurality of frequency bands are analyzed for all signal ranges from the minimum sampled rate to the maximum available frequency according to the Nyquist theorem (B/2), wherein B is a frequency at which the signal was sampled.

10. The method of claim 8, wherein higher frequency bands of interactions with electrocardiogram (ECG) signals of frequencies greater than a traditional 0 to 2 Hz band are given higher weight in quantification of the directed interactions.

11. The method of claim 8, wherein the model is a vector autoregressive model (VAR) or a multivariate auto-regressive model (MVAR), and
wherein the order of the model p is a pre-determined value or an optimally determined value.

12. The method of claim 1, wherein the impending crisis is related to an occurrence of a dynamical disorder of at least one of a brain, a heart or lungs,
wherein the occurrence of the dynamical disorder is an epileptic seizure, conditions of status epilepticus (SE), sudden infant death syndrome (SIDS), sudden unexpected death in epilepsy (SUDEP), a myocardial infarction, a complication or crises related to diabetes, respiratory apnea, sleep apnea, restless legs syndrome, narcolepsy, insomnia, cardiac arrest, neurodegeneration related to Parkinson's disease, neurodegeneration related to Alzheimer's disease, Parkinson's disease, stroke, and sudden arrhythmic death syndrome.

13. The method of claim 1, wherein the one or more pathologies comprise functional and/or structural neuronal damage in an afferent and/or an efferent connection between the at least two organs and/or between regions within at least one of the at least two organs.

14. The method of claim 1, wherein the one or more pathologies comprise Type 1 diabetes, Type 2 diabetes, a cardiac arrhythmia, ventricular tachycardia, apnea, ineffective sigh reflex, traumatic brain injury, neurodegeneration, stroke, or epilepsy.

15. The method of claim 1, wherein the informative feedback for a treatment regime is provided by:
monitoring any changes in the pathology following administration of the treatment; and
suggesting continuation of the treatment upon signs of improvement or suggesting altering the treatment if the pathology remains unchanged.

16. A system comprising:
a non-transitory memory storing computer-executable instructions; and
a processor that executes the computer-executable instructions to at least:
receive data recorded by one or more recording devices from a patient, wherein the data is time varying and related to two or more organs of a neuro-cardio-respiratory system within the patient's body;
estimate directional interactions between the two or more organs within the patient's body over time via a mathematical analysis of the data to identify one or more pathologies in a network of the neuro-cardio-respiratory system; and
provide an advance warning of an impending crisis, a diagnosis of the abnormality, or informative feedback for a treatment regime when the one or more pathologies in the network of neuro-cardio-respiratory system are identified.

17. The system of claim 16, wherein the processor identifies the one or more pathologies by quantifying at least one biomarker for the one or more pathologies.

18. The system of claim 16, further comprising the one or more recording mechanisms,
wherein the one or more recording mechanisms comprise at least one sensor.

19. The system of claim 16, wherein a treatment is prescribed in response to the warning.

20. The system of claim 16, wherein the directional interactions are estimated by:
determining an information inflow based on an analysis of the data from the organs, wherein the information inflow reflects a directional flow of information to each of the organs from at least one other organ.

21. The system of claim 20, wherein the determining the information inflow is conducted using at least one estimation method,
wherein the at least one estimation method comprises at least one of Coherence, Directed Coherence (DC), Partial Directed Coherence (PDC), Generalized Partial Directed Coherence (GPDC), Directed Transfer Function (DTF), Transfer Entropy, Mutual Information, Cross Correlation, Dynamical Entrainment, Non-Linear Interdependence, directed measures of Centrality, Phase Synchronization, Phase Amplitude Coupling, Bispectrum, and Bicoherence.

22. The system of claim 20, wherein the determining the information inflow comprises:
fitting a model of order p to successive segments of the data; and
determining a measure of inflow from each of the at least two organs from the at least one other organ in a plurality of frequency bands,
wherein the determining is conducted using Directed Coherence (DC), Partial Directed Coherence (PDC), Generalized Partial Directed Coherence (GPDC), or Directed Transfer Function (DTF).

23. The system of claim 22, wherein the plurality of frequency bands are analyzed for all signal ranges from the minimum sampled rate to the maximum available frequency according to the Nyquist theorem (B/2), wherein B is a frequency at which the signal was sampled.

24. The system of claim 22, wherein higher frequency bands of interactions with electrocardiogram (ECG) signals in higher than a traditional ECG frequency band of 0 to 2 Hz are given higher weight in quantification of the directed interactions.

25. The system of claim 20, wherein the model is a vector autoregressive model (VAR) or a multivariate auto-regressive model (MVAR), and
wherein p is a pre-determined value or an optimally determined value.
